(19) United States
(12) Patent Application Publication
(10) Pub. No.: US 2013/0216653 A1
(43) Pub. Date: Aug. 22, 2013

(54) SPORE SURFACE DISPLAY OF BIOACTIVE MOLECULES

(71) Applicants: John B. Perkins, Basel (CH); Zoltan Pragai, Basel (CH); Ghislain Schyns, Basel (CH)

(72) Inventors: John B. Perkins, Basel (CH); Zoltan Pragai, Basel (CH); Ghislain Schyns, Basel (CH)

(73) Assignee: DSM IP ASSETS B.V., Heerlen (NL)

(21) Appl. No.: 13/807,804

(22) PCT Filed: Jun. 28, 2011
(86) PCT No.: PCT/EP2011/060828

§ 371 Date: Mar. 18, 2013

(30) Foreign Application Priority Data
Jun. 30, 2010 (CH) 01068/10

(51) Int. Cl. C12N 11/16; A23K 1/00; C12N 15/74

(52) U.S. Cl. CPC C12N 11/16; C12N 15/74; A23K 1/009
USPC 426/61; 435/252.31; 435/252.3; 435/174

(57) ABSTRACT

The present invention relates to the display of bioactive molecules at the surface of spores for both in vitro and in vivo applications. Three small open reading frames (ORFs) have been identified which are very useful for display of bioactive molecules at the spore surface. The encoded small proteins have a molecular weight of less than 12 kDa, which corresponds to about less than 100 amino acids.

The present invention relates to the display of bioactive molecules at the surface of spores for both in vitro and in vivo applications.

During the last ten years microbial surface display (part of the bio-nanotechnology field) has increasingly become a tool of choice to display peptides or proteins of biotechnological interest on natural nanostructures for a commercial purpose. Biological applications include the development of bio-adsorbents, the presentation of antigens for vaccines, or the preparation of combinatorial epitope libraries. Surface display requires only the synthesis of a hybrid protein that consists of a passenger protein of commercial interest fused to a carrier protein, which anchors it onto the biological surface (cell wall or membrane). A good carrier protein requires the following characteristics: i) a targeting signal that directs it to the biological surface; ii) a strong anchoring motif; iii) resistance to proteases; and iv) compatibility to foreign sequences to be fused. Originally, the carrier protein was chosen amongst surface or membrane proteins, e.g. OmpA for Gram-negative bacteria or the Protein A for Gram-positive bacteria. The disadvantages of these display systems are that these proteins were not very stable and tended to be inactivated under conditions that are regularly used in biotechnological and chemical processes.

Recently, another nanostructure has emerged as a novel surface of choice for display: the spore coat from Bacillus subtilis and other related genera. Bacilli and Clostridia have the ability to undergo a complex differentiation process under nutrient deprivation or hostile conditions. This process, called sporulation, ends with the formation of an extremely resistant structure named the spore. When conditions become conductive for growth, the spores germinate to re-generate vegetative cells which follow a classical growth and division cyclic pattern. A spore consists of a central compartment, the spore core, which contains a copy of the chromosome. The spore core is surrounded by a thin inner layer membrane of peptidoglycan that creates the germ cell, itself surrounded by a thicker layer of peptidoglycan, called the cortex. Outside of the cortex, a multilayered protein shell, the coat, provides unique resistance characteristics. The B. subtilis coat is formed by the ordered assembly of over 40 polypeptides. Some of these have enzymatic activity, like oxdD, which encodes an oxalate decarboxylase, cotA which encodes a laccase, yvdO which encodes a phospholipase, cotQ which encodes a reticuline-oxidase or tgl which encodes a transglutaminase. In contrast to vegetative cells, the spore coat proteins allow spores to be very resistant to harsh chemicals, desiccation, strong pressure, or high temperatures. An example of B. subtilis spore is disclosed in WO 2005/028556. Known spores which show synthetic enzymatic activity displayed at the spore surfaces are very limited and refer to the use as diagnostic system or pharmaceutical drug, e.g. vaccine delivery systems. Examples reported are displays of β-galactosidases, which were fused to part of CotC, CotD, CotE, CotG or InhA (WO 1996/23063; WO 02/46388; WO 2005/028654), and displays of lipases, which were inserted in frame within CotC or fused to part of CotC (WO 02/00232) or displays of carboxymethylcellulases, which were fused to the exosporium protein InhA.

The spore surface proteins used so far as carriers for display of bioactive molecules have a molecular weight of at least 12 kDa, such as 12 (CotC) to 65 kDa (CotA). Carrier proteins having such weight/size turned out to be disadvantageous due to different types of interference with either the spore assembly and the spore structure or potentially with the folding of the passenger aimed to be displayed. If using such kind of carriers, there is a high risk of potential multimerization of passengers fused to the carrier, which would lead to display of multimeric bioactive molecules such as e.g. enzymes. Furthermore, the spore structure might be altered by such big carriers fused to the respective passenger.

Thus, it is desirable to look for smaller carriers as the ones known in the art which do not exhibit these negative effects and which could be used for displaying bioactive molecules on the spore surface.

Surprisingly, we now identified 3 small open reading frames (ORFs) which are very useful for display of bioactive molecules at the spore surface. The encoded small proteins have a molecular weight of less than 12 kDa, which corresponds to about less than 100 amino acids. The 3 ORFs have been identified/isolated from the intergenic regions of Bacillus subtilis or are paralogs of small proteins identified in the intergenic regions of Bacillis subtilis.

As used herein, a “small protein” or “carrier” is a protein displayed on the coat of the forespore and exhibits a molecular weight below about 12 kDa, corresponding to approximately 100 amino acids or less. It is encoded by a small transcription unit in the intergenic region of the genome of a suitable spore-forming microorganism, such as e.g. Bacilli, Sporolactobacilli and Clostridia, preferably Bacillus, more preferably B. subtilis.

Thus, the present invention is directed to a carrier/small protein and the DNA encoding said carrier used for displaying a bioactive molecule, wherein the carrier has the following properties:
(a) being a small protein of less than about 12 kDa corresponding to about 100 amino acids or less, preferably about 50 to 100 amino acids,
(b) being displayed at the surface of the forespore of a spore-forming microorganism, preferably selected from Bacilli, Sporolactobacilli and Clostridia, more preferably Bacillus, most preferably B. subtilis, and
(c) being encoded by a DNA which is under control of sporulation transcription factor sigma^{K}.

In particular, the carrier is selected from proteins of about 100 amino acids or less, such as between 50 and 100 amino acids, preferably about 50, 60, 70, 80, 90, 100 amino acids. More preferably, the ORFs are selected from ynzSP (FIG. 1), ydgB (FIG. 7) or ydzH (FIG. 8) coding for small proteins/carriers represented by SEQ ID NO:10 (YnzSP), SEQ ID NO:11 (YdgB), and SEQ ID NO:12 (YdzH). The ynzSP ORF has been identified in the intergenic region in the genome of B. subtilis. Both ydgB and ydzH are paralogs of sequences identified in the intergenic regions of the B. subtilis genome.

In one aspect, the present invention is directed to a construct comprising a first DNA encoding the carrier as specified above and a second DNA encoding the bioactive molecule, also referred herein as the “passenger”, wherein the carrier-passenger is expressed as a fusion protein. Thus, the present invention is directed to a fusion protein comprising (1) a carrier selected from YdgB, YdzH or YnzSP and (2) the passenger, selected from, e.g., proteins, enzymes or bioactive (poly)peptides. The use of such a construct as well as the fusion protein for display of bioactive molecules at the spore surface, the genetically modified spore itself as well as a microorganism comprising such a spore is also covered by the present invention.

Thus, the bioactive molecules or passengers to be displayed at the spore surface include but are not limited to proteins, enzymes, bioactive (poly)peptides such as e.g. bacteriocins, epitopes used for vaccination or affinity ligands that could bind the spore to the gut epithelium and anchor a spore which would have other bioactive molecules displayed.

Preferred enzymes useful as passengers and fused to one of the carriers mentioned above are any enzymes used in food or feed industry, in particular phytase (EC 3.1.3.8 or 3.1.3.26), xylanase (EC 3.2.1.8), galactanase (EC 3.2.1.89), alpha-galactosidase (EC 3.2.1.22), protease (EC 3.4.), phospholipases, beta-glucuronidase (EC 3.2.1.31), alkaline phosphatase, amylase such as, for example, alpha-amylase (EC 3.2.1.1) or beta-glucanase (EC 3.2.1.4 or EC 3.2.1.6). Examples of phospholipases are phospholipase Al (EC 3.1.1.32), phospholipase A2 (EC 3.1.1.4), lysophospholipase (EC 3.1.1.5), phospholipase C (EC 3.1.4.3) or phospholipase D (EC 3.1.4.4).

In a particular embodiment, the spores according to the present invention comprise YdzH, YnzSP or YdgB fused to phytase, alkaline phosphatase, beta-glucoronidase, green fluorescence protein or affinity ligands such as, e.g., PexS.

Soluble enzymes can be immobilized following different procedures mainly in order to reuse and to stabilize them. Examples of immobilized enzymes are Candida rugosa lipase (CRL) encapsulated without carrier, trypsin, Candida Antarctica lipase (CalB) or penicillin G acylase cross-linked to macromolecule (e.g. polyethylene glycol or dextran sulfate) or alkylsulfatase on anionic exchangers.

An example of a useful passenger is the A. niger PTS-1 affine PexS protein. PexS is the receptor of PTS-1 [McCollum et al., J. Cell Biol. 121, 761-774 (1993)]. PTS-1 is a C-terminal tri-peptide extension of a protein promoting peroxisomal localization of the protein. The C-terminal tri-peptide PTS-1 can be a variant of [PAS]-[HKR]-[L] as described in Emanuelsson et al., J. Mol. Biol. (2003) 330, 443-456. Preferably PTS-1 is -SKL or -PRL. The term “affinity ligand” as used herein denotes not only molecules that have biological relationship in vivo with the target protein but also a variety of other ligands such as fusion proteins or affinity tags. Examples of affinity tags or fusion proteins are the maltose binding protein (MBP) that interacts with cross-linked amylose and is eluted with maltose, polyhistidine tags that consists of 6 His residues binding to chelated Ni²⁺ or FLAG tag that is an eight amino acid hydrophilic peptide that binds to a specific antibody linked onto a column.

Suitable bioactive (poly)peptides which can be used as a passenger fused to one of the carriers mentioned above are antimicrobial and/or antifungal polypeptides. Examples of antimicrobial peptides (AMP's) are CAP18, leucocin A, tritrpticin, protegrin-1, thanatin, defensin, lactoferrin, lactoferricin, and ovispirin such as novispirin, plectasins, and statins, including the compounds and polypeptides disclosed in WO 03/044049 and WO 03/048148, as well as variants or fragments of the above that retain antimicrobial activity. Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

It is another object of the present invention to provide a genetically modified, viable spore which is able to germinate, wherein said spore is genetically modified to produce an enzyme or a bioactive polypeptide upon germination into a vegetative cell, said enzyme or bioactive polypeptide as defined above being displayed on the spore surface.

Inert spores are spores which are unable to germinate and recreate vegetative life. Methods to generate Bacillus subtilis non-germinating strains are well known from people skilled in the art. Inert spores according to this aspect of the invention are for example used “in vitro” and allow for example an alternative option to expensive classical systems of immobilized enzymes. They primarily have the advantage of spore resistance to harsh chemical conditions.

Such genetically modified or genetically engineered viable spore systems expressing bioactive molecules at the spore surface have a great potential use in particular in animal feeding. Further, it has been found that genetically modified or “genetically engineered” inert spore systems expressing affinity ligands or immobilized enzymes at the surface have a great potential use in biocatalysis and in downstream purification processes. Especially the resistance to harsh chemicals, desiccation, strong pressure, or high temperatures allows the spores to be a potentially valuable tool for the display of bioactive molecules, like biocatalytic enzymes or bioactive feed enzymes that must survive harsh reaction conditions to deliver their full potential.

Thus, it is an object of the present invention to provide a new genetically modified, inert spore which is unable to germinate, wherein said spore is genetically modified to expose at its surface affinity ligands, enzymes, such as e.g. immobilized enzymes, or epitopes.

The terms “spore” and “spore system” as used herein are equivalent expressions and denote differentiated resistant structures that come from differentiation of microbial vegetative cells under hostile physical or chemical conditions such as, but not limited to, extreme pH, heat, pressure, desiccation or an extract/mixture containing said structures, wherein the spore is derived from a parent spore-forming organisms.

The spore which can be used in the present invention may be publicly available from different sources, e.g., Deutsche Sammlung von Mikroorganismen and Zellkulturen (DSMZ), Inhoffenstrasse 7B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazu-shi, Chiba, 292-0818, Japan (formerly: Institute for Fermentation, Osaka (IFO), 17-85, Juso-honmachi 2-chome,Yodogawa-ku, Osaka 532-8686, Japan), or alternatively from well characterized (wild) isolates, which sporulate with higher efficiency than laboratory strains. Examples of preferred spores are spores of Bacilli, Sporolactobacilli and Clostridia, for example bacterial spores of B. subtilis.

The term “genetically modified” or “genetically engineered” means the scientific alteration of the structure of genetic material in a living organism. It involves the production and use of recombinant DNA. Genetic engineering may be done by a number of techniques known in the art, such as gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

In a particular aspect of the present invention, the genetically modified, inert spore comprises a recombinant DNA construct comprising a first DNA portion encoding the carrier and a second DNA portion encoding the passenger, which construct is expressed as a carrier-passenger fusion protein. The spore-forming microorganism expressing said fusion protein is preferably selected from Bacillus, more preferably from B. subtilis. Particular carrier-passenger combinations are mentioned above.

In a further aspect the invention relates to the use of inert spore systems expressing the passengers as described above. Particularly, the spore system as described herein is suitable for any enzymes used in the food or feed industry.

Display on viable/live spores allows amplification of spore population in situ through the sporulation-germination-vegetative growth cycle. Therefore, such a spore system according to the invention allows a continuously deliver of fresh enzymes or bioactive polypeptides. It is a further advantage of such systems that the spores are resistant to difficult conditions of digestive tracts and that they are easy to produce and can be made at low costs.

In a preferred embodiment of the invention, the genetic modification is accomplished by transformation of a precursor cell using a vector containing the chimeric transcription unit (chimeric DNA encoding the carrier/passenger fusion protein), using standard methods known to persons skilled in the art and then inducing the precursor cell to produce spores according to the invention. Further, the system may be constructed as such, that the chimeric DNA may be under the control of one or more inducible promoter. The chimeric construct may have one or more enhancer elements or upstream activator sequences and the like associated with it. The construct may also comprise an inducible expression system. The inducible expression system is such that when said spore germinates into a vegetative cell, the active polypeptide or enzyme is not expressed unless exposed to an external stimulus, e.g., change to a specific pH.

The DNA constructs encoding the carrier-passenger fusion protein to be displayed on the spore surface can be generated by methods known to the skilled person, wherein the carrier DNA is selected from ynzSP, ydgB or ydzH. It is not critical which one of the small proteins is fused to the passenger. Furthermore, any passenger described above, e.g. enzyme, affinity ligands, bioactive polypeptides or epitopes can be combined with said carrier. It is also possible to display more than one carrier-passenger couple on the same time on the spore surface.

Examples of enzymes displayed on the spore surface and used as carrier are alkaline phosphatase (PhoA), beta-glucuronidase (GUS) or phytase (Phy) which can be fused to one of the carriers, such as e.g. YnzSP, YdgB or YdzH. Translational fusions are generated using the carrier (e.g. ynzSP, ydgB or ydzH) and the passenger DNA (e.g. phoA, uidA gene of E. coli or phy). These constructs are then cloned into the BamHI-HindIII restriction site of a suitable vector, such as e.g. the B. subtilis suicide vector pDG364 (BGSC-ECE46; Karmazyn-Campelli et al., 1989). The resulting plasmid is then linearized, e.g. using XhoI, and transformed by double-crossover recombination at the non-essential amyE locus into a suitable strain, such as e.g. B. subtilis PY79. The resulting strain can be used for spore display. The respective translational fusions are shown in FIGS. 1 to 3.

An example of a spore displaying an affinity ligand is as follows: the Aspergillus niger pex5 gene encodes for a protein which is recognizing specifically PTS-1 motifs [e.g. SKL (serine-lysine-leucine) motifs or PRL (proline-arginine-leucine)]. The PTS-1 motif can be engineered at the carboxyl-terminus of protein for specific tagging and subsequent capture of the tagged protein. A translational fusion using ynzSP and pex5 can be generated as described above, whereby the construct is cloned into the BamHI-HindIII restriction site of a suitable vector, such as e.g. the B. subtilis suicide vector pDG364 (BGSC-ECE46; Karmazyn-Campelli et al., 1989). The resulting plasmid is then linearized, e.g. using XhoI, and transformed by double-crossover recombination at the non-essential amyE locus into a suitable strain, such as e.g. B. subtilis PY79.

In order to improve expression of the affinity ligand, the A. niger pex5 coding sequence (passenger sequence, underlined in FIG. 4) may be codon-adapted for expression in B. subtilis. The relevant optimized passenger sequence, which is designed to be free of BamHI, HindIII and Nhel sites, is detailed in FIG. 5 and strictly encodes the same protein as the passenger sequence of FIG. 4 (FIG. 6). The ynzSP-ala10(NheI)-optipex5 synthetic translational fusion is subsequently cloned between the BamHI and HindIII sites into the B. subtilis suicide vector pDG364 for ectopic integration within the non-essential amyE locus and transformed into a suitable strain, such as e.g. B. subtilis PY79.

The construction of a strain to display green fluorescence protein (GFP) fused to YdzH is described in Example 1 (see FIG. 9).

Display of enzymatic activity or activity of the affinity ligand can be measured by known techniques, such as described in WO 2008/017483 (see in particular the Examples).

If the spore system according to the invention expresses a feed enzyme on the spore surface, the spore germinates in the intestinal tract. More preferably, the spore germinates in the duodenum and/or the jejunum of the intestinal tract.

In a further aspect of the invention the viable spore can be constructed as such that it displays a combination of bioactive molecules, such as e.g., an enzyme, such as e.g. a feed enzyme, and a bioactive polypeptide.

It is a further object of the invention to provide a composition comprising spores which express bioactive molecules as defined herein on their surface. The bioactive molecule may be an enzyme, bioactive (poly)peptides, an epitope and/or an affinity ligand. Thus, the composition may comprise a spore expressing an enzyme and a bioactive polypeptide as passenger on the spore surface. Particularly, the composition comprises spores of the invention which express a feed enzyme, preferably phytase (EC 3.1.3.8 or 3.1.3.26), beta-glucuronidase (EC 3.2.1.31) or alkaline phosphatase. A composition according to the present invention may comprise a spore system expressing affinity ligands such as e.g. A. niger PTS-1-affine PexS protein or the green fluorescence protein (GFP).

In a further aspect, the compositions of the invention comprising a spore system as described herein are used in the feed industry as e.g. additive to animal feed. Said animal feed compositions may comprise a spore expressing a feed enzyme according to the invention and at least one or more vitamins and further compounds used in animal feeding and known to the skilled person. The vitamins may be either water- or fat-soluble. Furthermore, the animal feed composition may have a crude protein content of 50 to 800 g/kg and comprise a spore expressing a feed enzyme according to the invention.

The term feed or feed composition means any compound, preparation, mixture, or composition suitable for, or intended for intake by an animal. The animal feed composition comprising the spore system as of the present invention may be available in the form of a premix.

Examples of animals are non-ruminants, and ruminants. Ruminant animals include, for example, animals such as sheep, goat, and cattle, e.g. cow such as beef cattle and dairy cows. In a particular embodiment, the animal is a non-ruminant animal. Non-ruminant animals include mono-gastric animals, e.g. pig or swine (including, but not limited to, piglets, growing pigs, and sows); poultry such as turkeys, ducks and chickens (including but not limited to broiler chicks, layers); fish (including but not limited to salmon, trout, tilapia, catfish and carp); and crustaceans (including but not limited to shrimp and prawn). The term animal does not include a human being.

The composition may further comprise feed-additive ingredients such as coloring agents, e.g. carotenoids such as beta-carotene, astaxanthin, and lutein; aroma compounds; stabilizers, antimicrobial peptides, polyunsaturated fatty acids and/or reactive oxygen generating species.

In a particular embodiment, the animal feed additive of the invention is intended for being included (or prescribed as having to be included) in animal diets or feed, in particular in premixes, at levels of 0.01 to 10.0%; more particularly 0.05 to 5.0%; or 0.2 to 1.0% (% meaning g additive per 100 g feed).

Animal feed compositions or diets have a relatively high content of protein. Poultry and pig diets can be characterized as indicated in Table B of WO 01/58275, columns 2-3. Fish diets can be characterized as indicated in column 4 of this Table B. Furthermore, such fish diets usually have a crude fat content of 200-310 g/kg. WO 01/58275 is hereby incorporated by reference.

Furthermore, or as an alternative to the crude protein content indicated above, the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein or protein source. It may also contain animal protein, such as Meat and Bone Meal, and/or Fish Meal, typically in an amount of 0-25%. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal.

In a particular embodiment, the vegetable protein source is material from one or more plants of the family Fabaceae, e.g. soybean, lupine, pea, or bean. In another particular embodiment, the vegetable protein source is material from one or more plants of the family Chenopodiaceae, e.g. beet, sugar beet, spinach or quinoa.

Other examples of vegetable protein sources are rapeseed, sunflower seed, cotton seed, cabbage and cereals such as barley, wheat, rye, oat, maize (corn), rice, triticale, and sorghum.

In still further particular embodiments, the animal feed composition of the invention contains 0-80% maize; and/or 0-80% sorghum; and/or 0-70% wheat; and/or 0-70% Barley; and/or 0-30% oats; and/or 0-30% rye; and/or 0-40% soybean meal; and/or 0-25% fish meal; and/or 0-25% meat and bone meal; and/or 0-20% whey.

Animal diets can e.g. be manufactured as mash feed (non pelleted) or pelleted feed.

Typically, the milled feed-stuffs are mixed and sufficient amounts of essential vitamins and minerals are added according to the specifications for the species in question. The spore strain can be added as solid or liquid formulation. It is at present contemplated that the Bacillus strain is administered in one or more of the following amounts (dosage ranges): 10 E2-14, 10 E4-12, 10 E6-10, 10 E7-9, preferably 10 E8 CFU/g of feed (the designation E meaning exponent, viz., e.g., 10 E2-14 means 102-1014).

The following embodiments are part of the invention:
(1) A spore which is genetically modified or genetically engineered by a genetic DNA construct, wherein the genetic DNA construct comprises a first DNA portion encoding a carrier and a second DNA portion encoding a passenger which is a bioactive molecule and, which construct, when transcribed and translated, expresses a fusion protein between the carrier and the passenger.
(2) A spore as above, which is a spore of Clostridia or Sporolactobacillus or Bacillus, preferably Bacillus subtilis, more preferably Bacillus subtilis 1A747.
(3) A spore as above, wherein the first DNA portion of the construct encoding the carrier is a small protein with a molecular weight of less than 12 kDa, which is displayed at the surface of the forespore of a spore-forming microorganism and which is encoded by a DNA which is under control of sporulation transcription factor sigma^{K}, preferably said small protein is selected from YnzSP (FIG. 10), YdgB (FIG. 11) or YdzH (FIG. 12).
(4) A spore as above, wherein the spore is a inert spore and unable to germinate wherein said spore is genetically modified to expose at their surface an affinity ligand and/or a biocatalyst, preferably an immobilized enzyme.
(5) A spore as above, wherein the spore is a viable spore which is able to germinate wherein said spore is genetically modified to produce a feed enzyme, preferably phytase, and/or a bioactive polypeptide, preferably bacteriocin, upon germination into a vegetative cell.
(6) A composition comprising spores as under (5).
(7). Use of a composition as under (6) as animal feed additive.
(8). Use of a spore strain as defined under 1 in the preparation of a composition for use in animal feed.
(9) A method for improving the feed conversion ratio (FCR), wherein a spore strain as defined under (5) is added to animal feed.
(10) An animal feed additive comprising (a) a spore strain as defined under (5), (b) at least one fat-soluble vitamin, and (c) at least one water-soluble vitamin.
(11) An animal feed composition having a crude protein content of 50 to 800 g/kg and comprising a spore strain as defined under (5).

### FIGURES

FIG. 1. Sequence of the ynzSP-(ala)15-phoA-SPfree translational fusion (SEQ ID NO: 1). BamHI and HindIII cloning sites are in bold underlined. The coding sequence of ynzSP is in bold. The coding sequence of phoA is underlined. Spacer region is in upper case font.

FIG. 2. Sequence of the ynzSP-(ala)15-phy-Sigfree translational fusion (SEQ ID NO:2). BamHI and HindIII cloning sites are in bold underlined. The coding sequence of ynzSP is in bold. The coding sequence of phy is underlined. Spacer region is in upper case font.

FIG. 3. Sequence of the ynzSP-ala10(NheI)-uidA synthetic translational fusion (SEQ ID NO:3). BamHI and HindIII cloning sites are in bold underlined. The coding sequence of ynzSP is in bold. The coding sequence of uidA is underlined. Spacer region is in lower case font. NheI restriction site in the spacer is in lower case underlined fonts.

FIG. 4. Sequence of the ynzSP-ala10-pex5 translational fusion (SEQ ID NO:4). BamHI and HindIII cloning sites are in bold underlined. The coding sequence of ynzSP is in bold. The coding sequence of pex5 is underlined. Spacer region is in lower case font.

FIG. 5. Sequence of A. niger pex5 coding sequence (SEQ ID NO:5), codon-adapted for expression in B. subtilis. Underlined TAATAA are stop codons.

FIG. 6. Amino acid sequence of the A. niger PexS protein (SEQ ID NO:6).

FIG. 7. Sequence of the ydgB gene (SEQ ID NO:7) encoding the mother-cell-specific sigma factor K-controlled spore-associated short protein identified as carrier. Sequence is in bold downstream from 200 by of the promoter sequence.

FIG. 8. Sequence of the ydzH gene (SEQ ID NO:8) encoding the mother-cell-specific sigma factor K-controlled spore-associated short protein identified as carrier. Sequence is in bold downstream from 200 by of the promoter sequence.

FIG. 9. Sequence of the ydzH-gfp translational fusion (SEQ ID NO:9). The coding sequence of ydzH is in bold. The coding sequence of gfp is underlined.

FIG. 10. Amino acid sequence of YnzSP (SEQ ID NO:10).

FIG. 11. Amino acid sequence of YdgB (SEQ ID NO:11).

FIG. 12. Amino acid sequence of YdzH (SEQ ID NO:12).

### EXAMPLES

General methodology concerning strains, plasmids, media, molecular and genetic techniques, spore purification, immunofluorescence detection, fluorescent detection of β-glucuronidase, β-glucuronidase (GUS) assay, alkaline phosphatase assay, phytase assay, activity assay, and photometric measurement of the released Pi (Alko method) are as described in WO 2008/017483 (page 11-14). All synthetic gene fusions are made commercially at DNA2.0 (Menlo park, Calif.).

### Example 1

Construction of B. subtilis Strain SD39 designed to Display Green Fluorescence Protein fused to YdzH

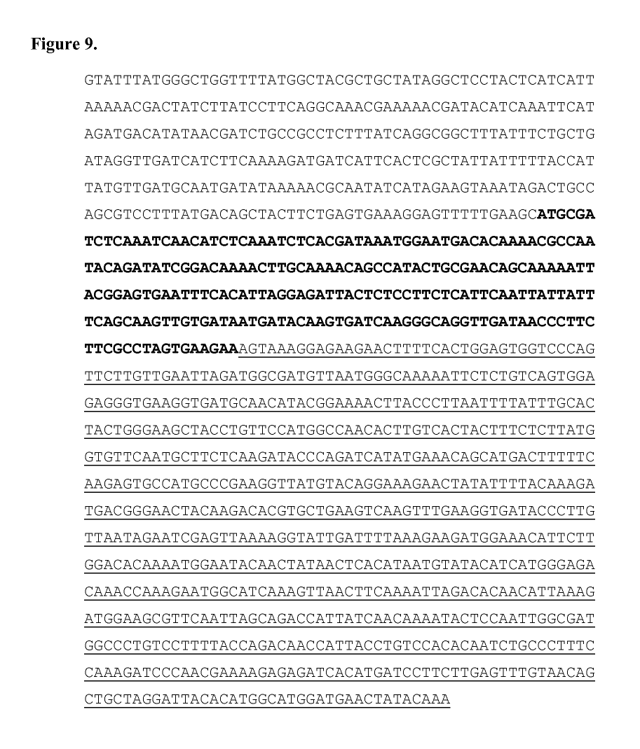

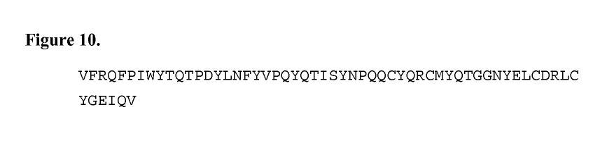

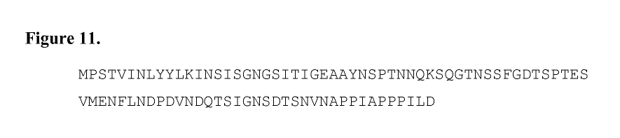

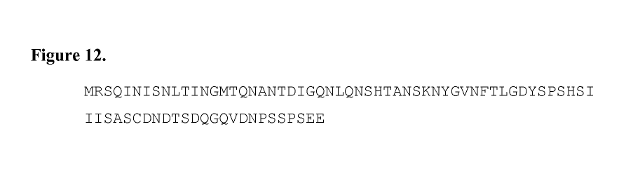

This example describes the construction of B. subtilis strain designed to display Green Fluorescence Protein (GFP) at the spore surface through fusion with the spore-associated short protein YdzH. The sequence of the ydzH-gfp translational fusion is given in FIG. 9. GFP is fused to the 3′ terminus of the ydzH.

The translational fusion is cloned between the BamHI and HindIII sites into a B. subtilis suicide vector pDG364 for subsequent ectopic integration within the non-essential amyE locus. The resulting plasmid is linearized with XhoI and transformed into B. subtilis PY79, resulting by double-crossover recombination at the non-essential amyE locus in a B. subtilis spore display strain.

A fluorescent micrograph of Bacillus subtilis cells expressing GFP fused to the 3′ terminus of the ydzH gene was generated. The cells were collected 4 h after induction of sporulation by resuspension and stained with a fluorescent dye. YdzH-GFP proteins form foci around the outside of the forespore.

### Example 2

Construction of B. subtilis Strains Designed to Display Enzymes Fused to YnzSP

Construction of the gene fusions is started by independent PCR amplifications of carrier and passenger fragments, subsequently combined by overlapping PCR to generate the translational fusions according to WO 2008/017483 except that ynzSP is used as carrier DNA.

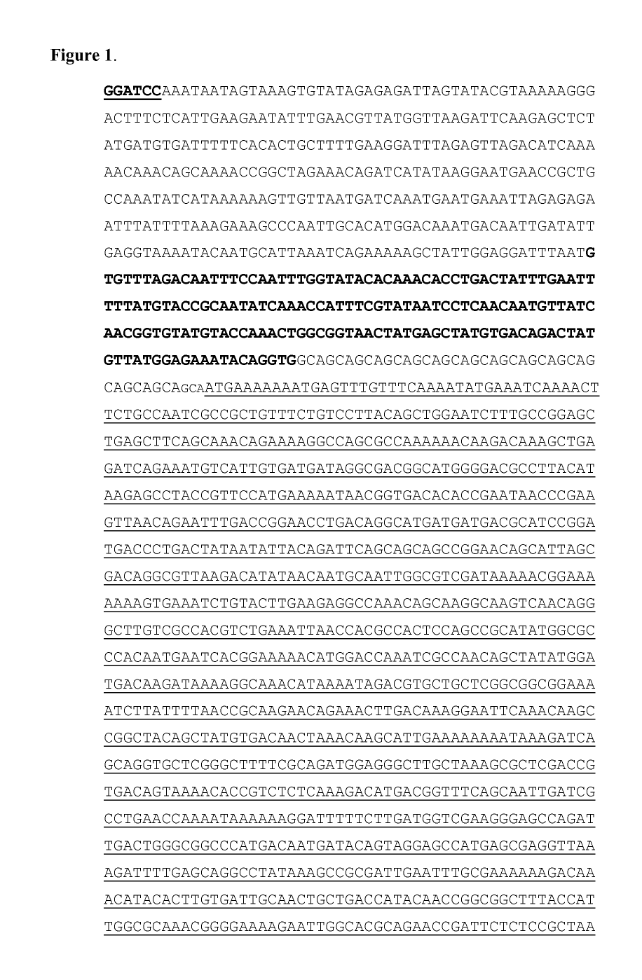

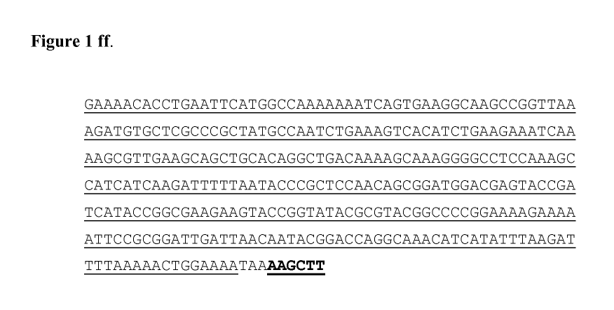

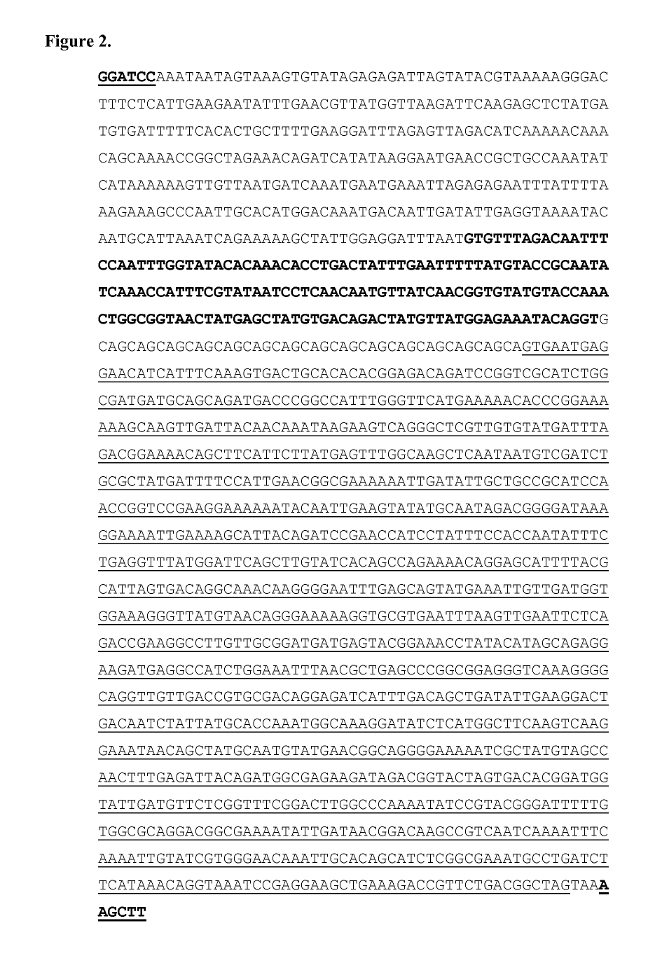

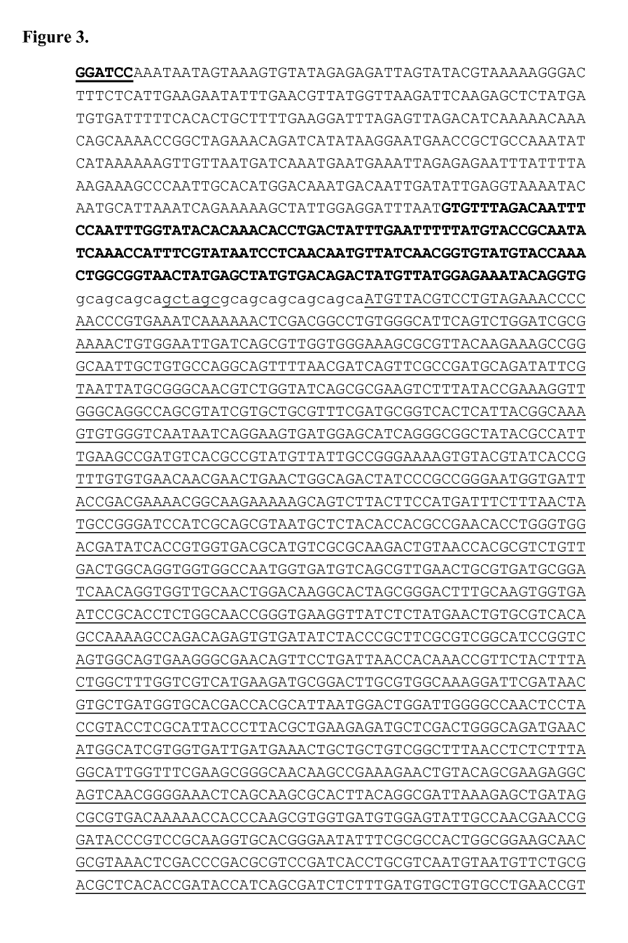

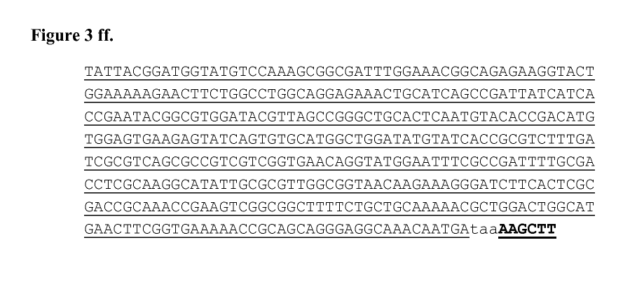

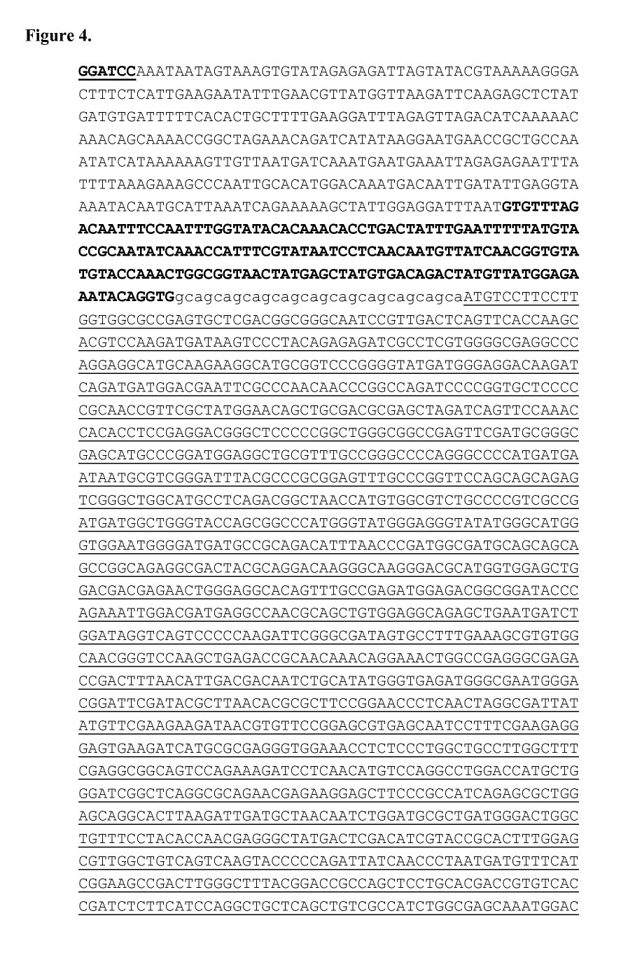

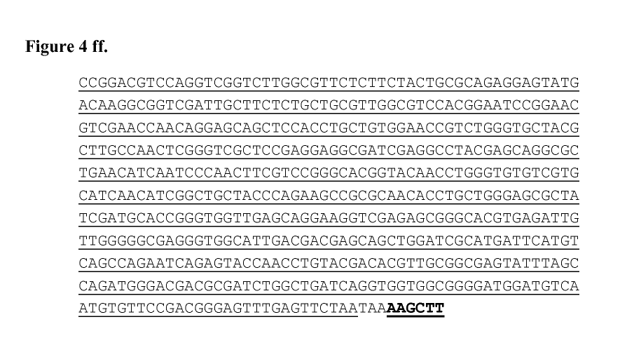

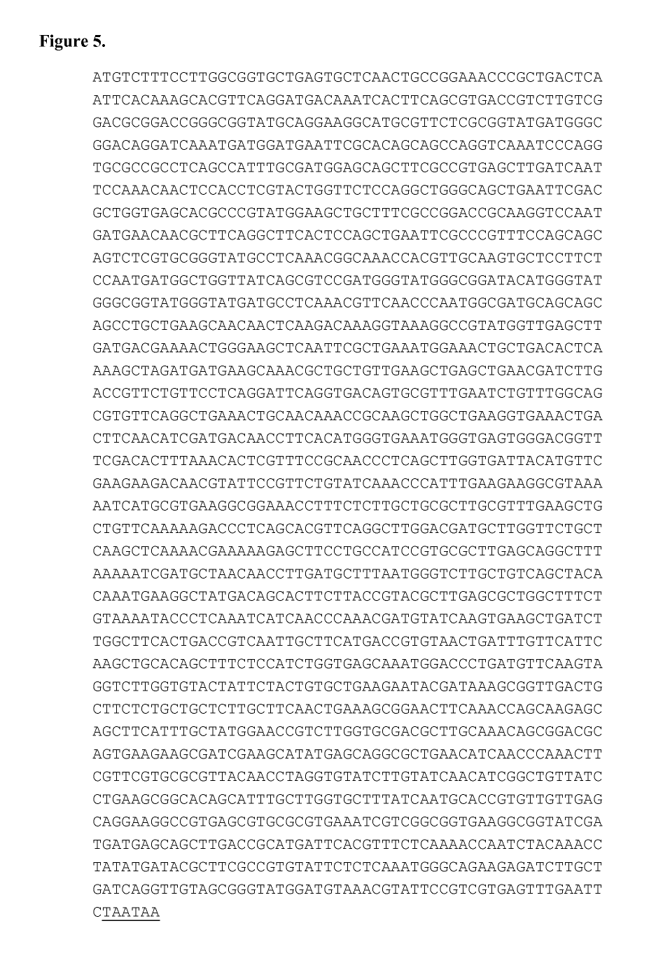

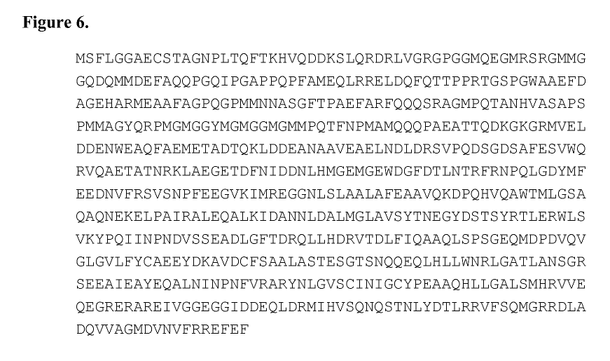

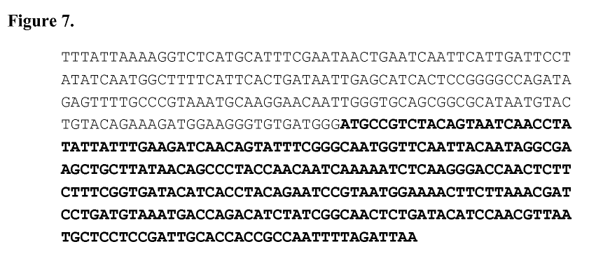

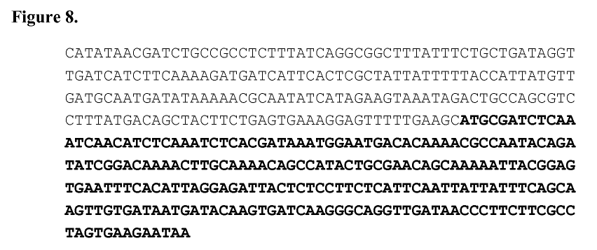

The enzymes selected as carriers are alkaline phosphatase, phytase, and β-glucuronidase, respectively. The respective translational fusions are shown in FIG. 1 (i.e. SEQ ID NO:1), FIG. 2 (i.e. SEQ ID NO:2) or FIG. 3 (i.e. SEQ ID NO:3), wherein the genes encoding said enzymes are fused to the 3′-end of the ynzSP ORF. The fusion constructs are cloned into the BamHI-HindIII restriction site of the B. subtilis suicide vector pDG364 (BGSC-ECE46; Karmazyn-Campelli et al., 1989). After linearization of the resulting plasmids with XhoI they are transformed into B. subtilis PY79 by double-crossover recombination into the amyE locus.

The analysis of the spores is performed as described in Example 1 or according to WO 2008/017483.

1. A spore of Bacillus or Clostridium or Sporolactobacillus which expresses a fusion protein, said fusion protein being expressed from a DNA construct comprising (i) a carrier DNA and (ii) a passenger DNA, wherein:
(i) the carrier DNA is under control of sporulation transcription factor sigma^{K} encoding a protein of less than 100 amino acids which is displayed at the surface of the forespore and
(ii) the passenger DNA is encoding a biocatalyst, a polypeptide or an affinity ligand.

2. A spore according to claim 1, wherein the carrier is selected from the group consisting of SEQ ID NO: 10, 11, and 12.

3. A spore according to claim 1, wherein the biocatalyst is selected from enzymes, preferably feed enzymes.

4. A spore according to claim 3, wherein the enzyme is immobilized.

5. A spore according to claim 1, wherein (i) the spore is unable to germinate exposing at the surface an affinity ligand and/or a biocatalyst or (ii) the spore is able to germinate producing a enzyme and/or a bioactive polypeptide upon germination into a vegetative cell.

6. A spore according to claim 1, wherein the fusion protein is selected from the group consisting of SEQ ID NO:1, 2, 3, and 4.

7. A spore according to claim 3, wherein the enzyme is phytase.

8. A composition comprising spores according to claim 1.

9. Use of a composition according to claim 8 as animal feed additive.

10. Use of a spore strain as defined in claim 1 in the preparation of a composition for use in animal feed.

11. A method for improving the feed conversion ratio (FCR), wherein a spore as defined in claim 3 is added to animal feed.

12. An animal feed additive comprising:
(a) a spore as defined in claim 3,
(b) at least one fat-soluble vitamin, and
(c) at least one water-soluble vitamin.

13. An animal feed composition having a crude protein content of 50 to 800 g/kg and comprising a spore as defined in claim 3.


SEQUENCE LISTING (12 sequences)

SEQ ID NO: 1 - DNA, 1964 bp, Artificial Sequence
  translational fusion
ggatccaaataatagtaaagtgtatagagagattagtatacgtaaaaagggactttctcattgaagaatatttgaacgttatggttaagattcaagagctctatgatgtgatttttcacactgcttttgaaggatttagagttagacatcaaaaacaaacagcaaaaccggctagaaacagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtggcagcagcagcagcagcagcagcagcagcagcagcagcagcaatgaaaaaaatgagtttgtttcaaaatatgaaatcaaaacttctgccaatcgccgctgtttctgtccttacagctggaatctttgccggagctgagcttcagcaaacagaaaaggccagcgccaaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgagcaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcagtgaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaataaaagctt

SEQ ID NO: 2 - DNA, 1648 bp, Artificial Sequence
  translational fusion
ggatccaaataatagtaaagtgtatagagagattagtatacgtaaaaagggactttctcattgaagaatatttgaacgttatggttaagattcaagagctctatgatgtgatttttcacactgcttttgaaggatttagagttagacatcaaaaacaaacagcaaaaccggctagaaacagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtgcagcagcagcagcagcagcagcagcagcagcagcagcagcagcagtgaatgaggaacatcatttcaaagtgactgcacacacggagacagatccggtcgcatctggcgatgatgcagcagatgacccggccatttgggttcatgaaaaacacccggaaaaaagcaagttgattacaacaaataagaagtcagggctcgttgtgtatgatttagacggaaaacagcttcattcttatgagtttggcaagctcaataatgtcgatctgcgctatgattttccattgaacggcgaaaaaattgatattgctgccgcatccaaccggtccgaaggaaaaaatacaattgaagtatatgcaatagacggggataaaggaaaattgaaaagcattacagatccgaaccatcctatttccaccaatatttctgaggtttatggattcagcttgtatcacagccagaaaacaggagcattttacgcattagtgacaggcaaacaaggggaatttgagcagtatgaaattgttgatggtggaaagggttatgtaacagggaaaaaggtgcgtgaatttaagttgaattctcagaccgaaggccttgttgcggatgatgagtacggaaacctatacatagcagaggaagatgaggccatctggaaatttaacgctgagcccggcggagggtcaaaggggcaggttgttgaccgtgcgacaggagatcatttgacagctgatattgaaggactgacaatctattatgcaccaaatggcaaaggatatctcatggcttcaagtcaaggaaataacagctatgcaatgtatgaacggcaggggaaaaatcgctatgtagccaactttgagattacagatggcgagaagatagacggtactagtgacacggatggtattgatgttctcggtttcggacttggcccaaaatatccgtacgggatttttgtggcgcaggacggcgaaaatattgataacggacaagccgtcaatcaaaatttcaaaattgtatcgtgggaacaaattgcacagcatctcggcgaaatgcctgatcttcataaacaggtaaatccgaggaagctgaaagaccgttctgacggctagtaaaagctt

SEQ ID NO: 3 - DNA, 2381 bp, Artificial Sequence
  translational fusion
ggatccaaataatagtaaagtgtatagagagattagtatacgtaaaaagggactttctcattgaagaatatttgaacgttatggttaagattcaagagctctatgatgtgatttttcacactgcttttgaaggatttagagttagacatcaaaaacaaacagcaaaaccggctagaaacagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtggcagcagcagctagcgcagcagcagcagcaatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgataaaagctt

SEQ ID NO: 4 - DNA, 2534 bp, Artificial Sequence
  translational fusion
ggatccaaataatagtaaagtgtatagagagattagtatacgtaaaaagggactttctcattgaagaatatttgaacgttatggttaagattcaagagctctatgatgtgatttttcacactgcttttgaaggatttagagttagacatcaaaaacaaacagcaaaaccggctagaaacagatcatataaggaatgaaccgctgccaaatatcataaaaaagttgttaatgatcaaatgaatgaaattagagagaatttattttaaagaaagcccaattgcacatggacaaatgacaattgatattgaggtaaaatacaatgcattaaatcagaaaaagctattggaggatttaatgtgtttagacaatttccaatttggtatacacaaacacctgactatttgaatttttatgtaccgcaatatcaaaccatttcgtataatcctcaacaatgttatcaacggtgtatgtaccaaactggcggtaactatgagctatgtgacagactatgttatggagaaatacaggtggcagcagcagcagcagcagcagcagcagcaatgtccttccttggtggcgccgagtgctcgacggcgggcaatccgttgactcagttcaccaagcacgtccaagatgataagtccctacagagagatcgcctcgtggggcgaggcccaggaggcatgcaagaaggcatgcggtcccggggtatgatgggaggacaagatcagatgatggacgaattcgcccaacaacccggccagatccccggtgctcccccgcaaccgttcgctatggaacagctgcgacgcgagctagatcagttccaaaccacacctccgaggacgggctcccccggctgggcggccgagttcgatgcgggcgagcatgcccggatggaggctgcgtttgccgggccccagggccccatgatgaataatgcgtcgggatttacgcccgcggagtttgcccggttccagcagcagagtcgggctggcatgcctcagacggctaaccatgtggcgtctgccccgtcgccgatgatggctgggtaccagcggcccatgggtatgggagggtatatgggcatgggtggaatggggatgatgccgcagacatttaacccgatggcgatgcagcagcagccggcagaggcgactacgcaggacaagggcaagggacgcatggtggagctggacgacgagaactgggaggcacagtttgccgagatggagacggcggatacccagaaattggacgatgaggccaacgcagctgtggaggcagagctgaatgatctggataggtcagtcccccaagattcgggcgatagtgcctttgaaagcgtgtggcaacgggtccaagctgagaccgcaacaaacaggaaactggccgagggcgagaccgactttaacattgacgacaatctgcatatgggtgagatgggcgaatgggacggattcgatacgcttaacacgcgcttccggaaccctcaactaggcgattatatgttcgaagaagataacgtgttccggagcgtgagcaatcctttcgaagagggagtgaagatcatgcgcgagggtggaaacctctccctggctgccttggctttcgaggcggcagtccagaaagatcctcaacatgtccaggcctggaccatgctgggatcggctcaggcgcagaacgagaaggagcttcccgccatcagagcgctggagcaggcacttaagattgatgctaacaatctggatgcgctgatgggactggctgtttcctacaccaacgagggctatgactcgacatcgtaccgcactttggagcgttggctgtcagtcaagtacccccagattatcaaccctaatgatgtttcatcggaagccgacttgggctttacggaccgccagctcctgcacgaccgtgtcaccgatctcttcatccaggctgctcagctgtcgccatctggcgagcaaatggacccggacgtccaggtcggtcttggcgttctcttctactgcgcagaggagtatgacaaggcggtcgattgcttctctgctgcgttggcgtccacggaatccggaacgtcgaaccaacaggagcagctccacctgctgtggaaccgtctgggtgctacgcttgccaactcgggtcgctccgaggaggcgatcgaggcctacgagcaggcgctgaacatcaatcccaacttcgtccgggcacggtacaacctgggtgtgtcgtgcatcaacatcggctgctacccagaagccgcgcaacacctgctgggagcgctatcgatgcaccgggtggttgagcaggaaggtcgagagcgggcacgtgagattgttgggggcgagggtggcattgacgacgagcagctggatcgcatgattcatgtcagccagaatcagagtaccaacctgtacgacacgttgcggcgagtatttagccagatgggacgacgcgatctggctgatcaggtggtggcggggatggatgtcaatgtgttccgacgggagtttgagttctaataaaagctt

SEQ ID NO: 5 - DNA, 1968 bp, Aspergillus niger
atgtctttccttggcggtgctgagtgctcaactgccggaaacccgctgactcaattcacaaagcacgttcaggatgacaaatcacttcagcgtgaccgtcttgtcggacgcggaccgggcggtatgcaggaaggcatgcgttctcgcggtatgatgggcggacaggatcaaatgatggatgaattcgcacagcagccaggtcaaatcccaggtgcgccgcctcagccatttgcgatggagcagcttcgccgtgagcttgatcaattccaaacaactccacctcgtactggttctccaggctgggcagctgaattcgacgctggtgagcacgcccgtatggaagctgctttcgccggaccgcaaggtccaatgatgaacaacgcttcaggcttcactccagctgaattcgcccgtttccagcagcagtctcgtgcgggtatgcctcaaacggcaaaccacgttgcaagtgctccttctccaatgatggctggttatcagcgtccgatgggtatgggcggatacatgggtatgggcggtatgggtatgatgcctcaaacgttcaacccaatggcgatgcagcagcagcctgctgaagcaacaactcaagacaaaggtaaaggccgtatggttgagcttgatgacgaaaactgggaagctcaattcgctgaaatggaaactgctgacactcaaaagctagatgatgaagcaaacgctgctgttgaagctgagctgaacgatcttgaccgttctgttcctcaggattcaggtgacagtgcgtttgaatctgtttggcagcgtgttcaggctgaaactgcaacaaaccgcaagctggctgaaggtgaaactgacttcaacatcgatgacaaccttcacatgggtgaaatgggtgagtgggacggtttcgacactttaaacactcgtttccgcaaccctcagcttggtgattacatgttcgaagaagacaacgtattccgttctgtatcaaacccatttgaagaaggcgtaaaaatcatgcgtgaaggcggaaacctttctcttgctgcgcttgcgtttgaagctgctgttcaaaaagaccctcagcacgttcaggcttggacgatgcttggttctgctcaagctcaaaacgaaaaagagcttcctgccatccgtgcgcttgagcaggctttaaaaatcgatgctaacaaccttgatgctttaatgggtcttgctgtcagctacacaaatgaaggctatgacagcacttcttaccgtacgcttgagcgctggctttctgtaaaataccctcaaatcatcaacccaaacgatgtatcaagtgaagctgatcttggcttcactgaccgtcaattgcttcatgaccgtgtaactgatttgttcattcaagctgcacagctttctccatctggtgagcaaatggaccctgatgttcaagtaggtcttggtgtactattctactgtgctgaagaatacgataaagcggttgactgcttctctgctgctcttgcttcaactgaaagcggaacttcaaaccagcaagagcagcttcatttgctatggaaccgtcttggtgcgacgcttgcaaacagcggacgcagtgaagaagcgatcgaagcatatgagcaggcgctgaacatcaacccaaacttcgttcgtgcgcgttacaacctaggtgtatcttgtatcaacatcggctgttatcctgaagcggcacagcatttgcttggtgctttatcaatgcaccgtgttgttgagcaggaaggccgtgagcgtgcgcgtgaaatcgtcggcggtgaaggcggtatcgatgatgagcagcttgaccgcatgattcacgtttctcaaaaccaatctacaaacctatatgatacgcttcgccgtgtattctctcaaatgggcagaagagatcttgctgatcaggttgtagcgggtatggatgtaaacgtattccgtcgtgagtttgaattctaataa

SEQ ID NO: 6 - protein, 654 aa, Aspergillus niger
MSFLGGAECSTAGNPLTQFTKHVQDDKSLQRDRLVGRGPGGMQEGMRSRGMMGGQDQMMDEFAQQPGQIPGAPPQPFAMEQLRRELDQFQTTPPRTGSPGWAAEFDAGEHARMEAAFAGPQGPMMNNASGFTPAEFARFQQQSRAGMPQTANHVASAPSPMMAGYQRPMGMGGYMGMGGMGMMPQTFNPMAMQQQPAEATTQDKGKGRMVELDDENWEAQFAEMETADTQKLDDEANAAVEAELNDLDRSVPQDSGDSAFESVWQRVQAETATNRKLAEGETDFNIDDNLHMGEMGEWDGFDTLNTRFRNPQLGDYMFEEDNVFRSVSNPFEEGVKIMREGGNLSLAALAFEAAVQKDPQHVQAWTMLGSAQAQNEKELPAIRALEQALKIDANNLDALMGLAVSYTNEGYDSTSYRTLERWLSVKYPQIINPNDVSSEADLGFTDRQLLHDRVTDLFIQAAQLSPSGEQMDPDVQVGLGVLFYCAEEYDKAVDCFSAALASTESGTSNQQEQLHLLWNRLGATLANSGRSEEAIEAYEQALNINPNFVRARYNLGVSCINIGCYPEAAQHLLGALSMHRVVEQEGRERAREIVGGEGGIDDEQLDRMIHVSQNQSTNLYDTLRRVFSQMGRRDLADQVVAGMDVNVFRREFEF

SEQ ID NO: 7 - DNA, 461 bp, Bacillus subtilis
tttattaaaaggtctcatgcatttcgaataactgaatcaattcattgattcctatatcaatggcttttcattcactgataattgagcatcactccggggccagatagagttttgcccgtaaatgcaaggaacaattgggtgcagcggcgcataatgtactgtacagaaagatggaagggtgtgatgggatgccgtctacagtaatcaacctatattatttgaagatcaacagtatttcgggcaatggttcaattacaataggcgaagctgcttataacagccctaccaacaatcaaaaatctcaagggaccaactcttctttcggtgatacatcacctacagaatccgtaatggaaaacttcttaaacgatcctgatgtaaatgaccagacatctatcggcaactctgatacatccaacgttaatgctcctccgattgcaccaccgccaattttagattaa

SEQ ID NO: 8 - DNA, 437 bp, Bacillus subtilis
catataacgatctgccgcctctttatcaggcggctttatttctgctgataggttgatcatcttcaaaagatgatcattcactcgctattatttttaccattatgttgatgcaatgatataaaaacgcaatatcatagaagtaaatagactgccagcgtcctttatgacagctacttctgagtgaaaggagtttttgaagcatgcgatctcaaatcaacatctcaaatctcacgataaatggaatgacacaaaacgccaatacagatatcggacaaaacttgcaaaacagccatactgcgaacagcaaaaattacggagtgaatttcacattaggagattactctccttctcattcaattattatttcagcaagttgtgataatgatacaagtgatcaagggcaggttgataacccttcttcgcctagtgaagaataa

SEQ ID NO: 9 - DNA, 1257 bp, Artificial Sequence
  translational fusion
gtatttatgggctggttttatggctacgctgctataggctcctactcatcattaaaaacgactatcttatccttcaggcaaacgaaaaacgatacatcaaattcatagatgacatataacgatctgccgcctctttatcaggcggctttatttctgctgataggttgatcatcttcaaaagatgatcattcactcgctattatttttaccattatgttgatgcaatgatataaaaacgcaatatcatagaagtaaatagactgccagcgtcctttatgacagctacttctgagtgaaaggagtttttgaagcatgcgatctcaaatcaacatctcaaatctcacgataaatggaatgacacaaaacgccaatacagatatcggacaaaacttgcaaaacagccatactgcgaacagcaaaaattacggagtgaatttcacattaggagattactctccttctcattcaattattatttcagcaagttgtgataatgatacaagtgatcaagggcaggttgataacccttcttcgcctagtgaagaaagtaaaggagaagaacttttcactggagtggtcccagttcttgttgaattagatggcgatgttaatgggcaaaaattctctgtcagtggagagggtgaaggtgatgcaacatacggaaaacttacccttaattttatttgcactactgggaagctacctgttccatggccaacacttgtcactactttctcttatggtgttcaatgcttctcaagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatgggagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctaggattacacatggcatggatgaactatacaaa

SEQ ID NO: 10 - protein, 58 aa, Bacillus subtilis
VFRQFPIWYTQTPDYLNFYVPQYQTISYNPQQCYQRCMYQTGGNYELCDRLCYGEIQV

SEQ ID NO: 11 - protein, 90 aa, Bacillus subtilis
MPSTVINLYYLKINSISGNGSITIGEAAYNSPTNNQKSQGTNSSFGDTSPTESVMENFLNDPDVNDQTSIGNSDTSNVNAPPIAPPPILD

SEQ ID NO: 12 - protein, 78 aa, Bacillus subtilis
MRSQINISNLTINGMTQNANTDIGQNLQNSHTANSKNYGVNFTLGDYSPSHSIIISASCDNDTSDQGQVDNPSSPSEE